(19) United States
(12) Patent Application Publication
(10) Pub. No.: US 2024/0115438 A1
(43) Pub. Date: Apr. 11, 2024

(54) MEDICAL SUPPLY DISPENSER FOR AMBULANCE UE

(71) Applicant: DONNUS GOVERNOR STRAUGHN, NEW ORLEANS, LA (US)

(72) Inventor: DONNUS GOVERNOR STRAUGHN, NEW ORLEANS, LA (US)

(21) Appl. No.: 18/134,267

(22) Filed: Apr. 13, 2023

Related U.S. Application Data
(62) Division of application No. 16/938,215, filed on Jul. 24, 2020, now abandoned.
(60) Provisional application No. 62/879,133, filed on Jul. 26, 2019.

(51) Int. Cl. A61F 15/00; B65D 6/00; B65D 83/00

(52) U.S. Cl. CPC A61F 15/001; B65D 11/00; B65D 83/0038

(57) ABSTRACT

Medical supply dispensers for ambulance use with a spring loaded dispenser for packaged bandages and a dispenser for flat intravenous IV bags, both intended for accessible mounting on the interior surfaces of an ambulance, within arm's reach of an Emergency Medical Technician.

### CROSS-REFERENCE TO RELATED APPLICATIONS

The present application is a Divisional application of, and claims priority under 35 USC § 121 to U.S. patent application Ser. No. 16/938,215, (the '215 application) filed on Jul. 24, 2020, entitled MEDICAL SUPPLY DISPENSER FOR AMBULANCE USE, and claiming priority from U.S. Provisional Application No. 62/879,133, filed Jul. 26, 2019. The '215 application is incorporated herein by reference in its entirety, for all purposes.

### BACKGROUND OF THE INVENTION

In Emergency Medical Services, saving time can mean saving lives. This is particularly true for first responders in an ambulance dealing with trauma. Ready and easy access to medical supplies is essential and can be problematic in the confines of a moving ambulance. The emergency medical techs (EMU) must quickly retrieve the bandages, intravenous supplies, medications and other equipment needed to deal with a patient. Included in those various devices and supplies are intravenous (IV) bags used to dispense fluids into the body of the patient. For example, in those situations where the patient is dehydrated, it is imperative that intravenous transfusion be started immediately and the EMT must reach for and select the required size of IV bag laid flat in a container and stored horizontally, one on top of another. This present arrangement makes what should be a simple task more difficult than it has to be. Likewise something as simple as reaching for a bandage can waste precious time if the EMT has to open a box of bandages or root through an open box of bandages to pull only one bandage. In addition, the boxes must be securely contained to withstand the vibration, bumps and motion associated with a fast moving ambulance.

### SUMMARY OF THE INVENTION

It is an object of this invention to provide a spring loaded dispenser for packaged bandages in ambulance use. The most commonly used bandage sizes are 4 inch by 4 inch and 2 inch by 2 inch, although this invention is suitable for any bandage size or shape, whether square or rectangular. The inventive dispenser would preferably have a plastic or other suitable non-metallic housing attached by any suitable method to a flat panel or other surface in the interior of an ambulance within arm's reach of an EMT dealing with a patient being transported in the ambulance. It is also intended that multiple packaged bandages be loaded in the dispenser through a dispenser opening against a moveable pusher plate causing compression of an internal spring placed between the rear end of the dispenser and the pusher plate. As the packaged bandages are removed from the dispenser opening one by one, the next available package is disposed and held in the dispenser opening in a convex shape against tabs at the top and bottom of the dispenser opening.

It is also an object of the present invention to offer a distinct improvement over the existing arrangement for EMTs to retrieve the properly sized IV bag one at a time without having to grope through a box of IV bags. In the present invention, IV bags are loaded flat into the top of a hollow dispenser of rectangular shape and dispensed one by one in a dispensing tray at the bottom of the dispenser. The hollow dispenser allows the IV bags to move downward under their own weight in the dispenser. The dispenser would be sized for a particular size of IV bag and mounted on a vertical surface in an ambulance within reach of the EMTs.

### BRIEF DESCRIPTION OF THE DRAWINGS

FIG. 1 is a perspective view of the inventive packaged bandage dispenser from the dispenser opening.

FIG. 2 is a cross sectional side view of the inventive packaged bandage dispenser fully loaded with packaged bandages.

FIG. 3 is a cross sectional side view of the inventive packaged bandage dispenser partially loaded with packaged bandages.

FIG. 4 is a perspective view of the inventive IV bag dispenser in the vertical position for mounting on a vertical surface in an ambulance or any treatment facility.

FIG. 5 is a view of a typical IV bag.

FIG. 6 is a front view of the inventive dispenser showing the dispenser tray and dispenser opening at the bottom end.

FIG. 7 is a cross sectional side view of the inventive dispenser showing the placement of IV bags inserted into the dispenser from the top end.

### DETAILED DESCRIPTION OF THE INVENTION

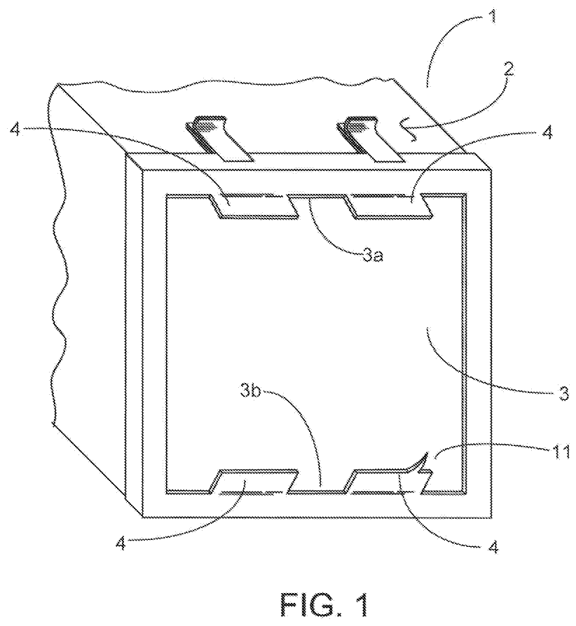

In FIG. 1, a perspective view of the inventive packaged bandage dispenser 1 shows the dispenser housing 2, with a rectangular dispenser opening 3 having a top edge 3a and a bottom edge 3b with at least one tab 4 on both the top edge 3a and bottom edge 3b. As can be seen the tabs 4 are bent outward from the dispenser 1. Also shown on one of the tabs 4 is a blade 11 to clip a corner of the packaged bandage 9 dispensed through the opening 3 to facilitate opening of the packaged bandage 9.

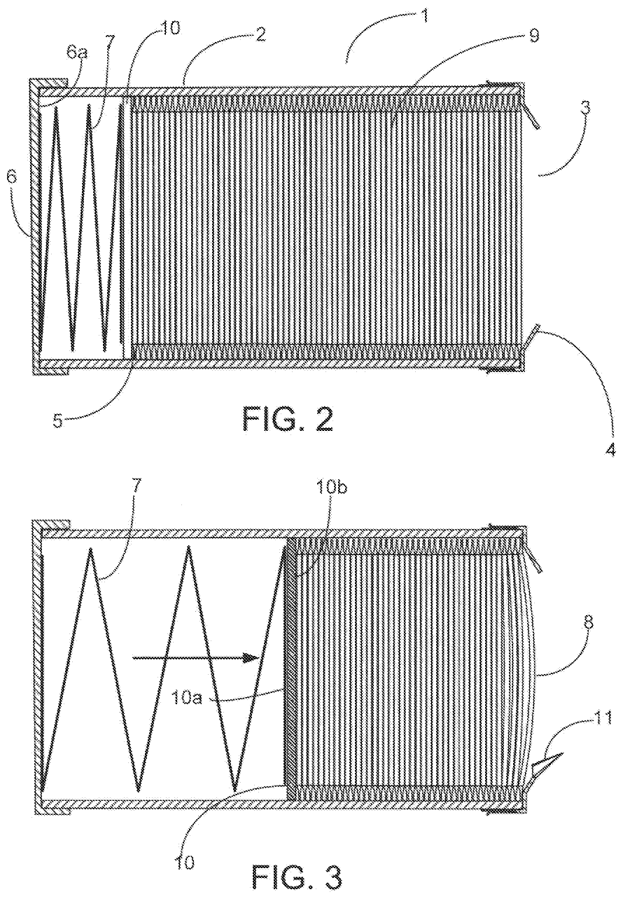

FIGS. 2 and 3 are both cross sectional side views of the inventive packaged bandage dispenser loaded with packaged bandages 9. FIG. 2 shows a fully loaded dispenser 1 with a fully compressed spring 7 while FIG. 3 shows a partially full dispenser with a partially extended spring 7. As shown in both FIGS. 2 and 3, the spring 7 pushes against the interior side 6a of the rear end 6 of the dispenser 1 and the spring side 10a of the pusher plate 10. The packaged bandage side 10b of the pusher plate 10 touches against a flat side of the first bandage 5 loaded into the dispenser. The stored packaged bandages 9 are manually removed one by one from the opening 3, with the next bandage 5 held in by the tabs 4 and exhibiting a slightly convex shape 8, making removal easier for the EMT.

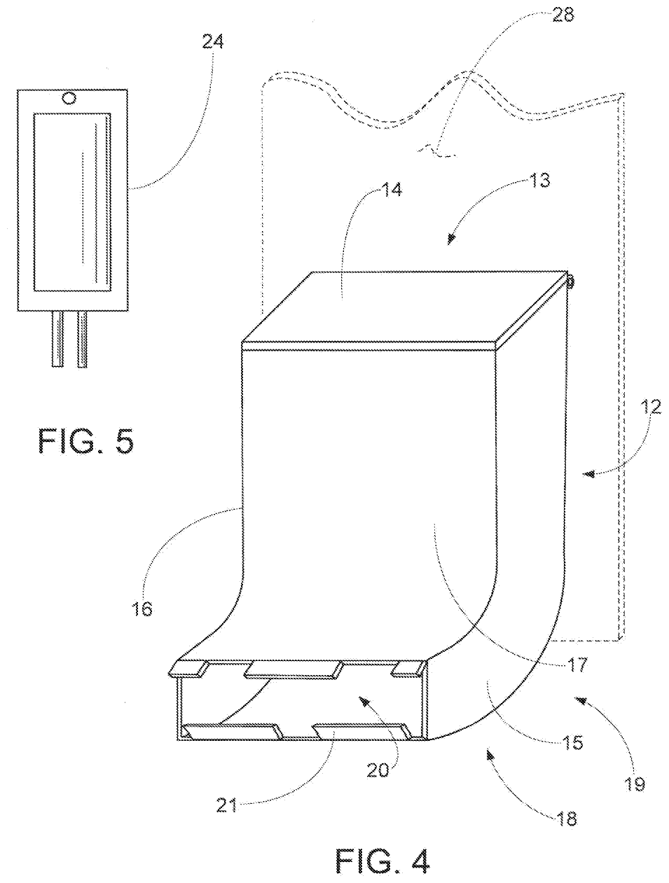

In FIG. 4, the inventive dispenser 12 is shown in a perspective view as it would be mounted on a vertical surface 28. The dispenser 12 has a top end 13, a top cover 14, a right side panel 15 a left side panel 16, a front face 17, a bottom end 18, a dispenser tray 19, with dispenser opening 20, and tray tabs 21. Although not fully seen in FIG. 4, there is a rear panel 22 that can only be seen on the inside surface 23 of the rear panel 22. As can be seen, the dispenser 12 is hollow with a rectangular configuration meant to receive IV bags 24 laid flat as depicted in FIG. 7.

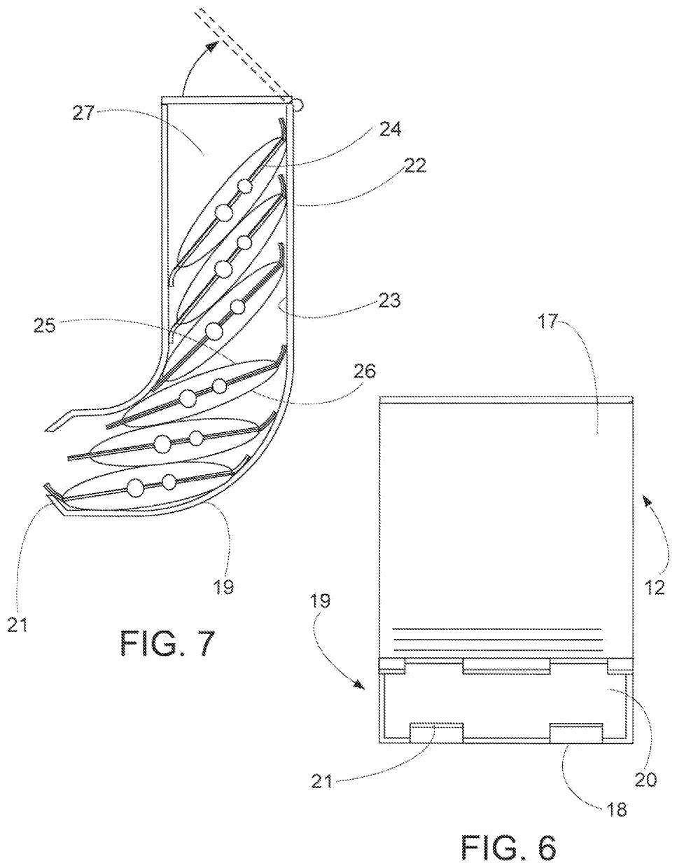

In FIG. 6, the inventive dispenser 12 is shown in a front view looking directly at the front face 17 with the top cover 14 shown on the top end 13 and the dispenser tray 19, dispenser opening 20 and tray tabs 21 shown at the bottom end 18.

FIG. 7 shows a cross sectional side view of the inventive dispenser 12 loaded with IV bags 24 each having a top flat side 25 and a bottom flat side 26. The bottom flat side 26 of the top bag is in contact with the top flat side 25 of the IV bag 24 directly below top bag and each successive bag in the hollow opening 27 of the dispenser 12. As can be seen in FIG. 7, the last

I claim:

1. A dispenser for packaged bandages in ambulance use securely attached to a surface in the interior of an ambulance within arm's reach of an EMT dealing with a patient being transported in the ambulance comprising:
a housing to store and dispense packaged bandages having a rear end with an interior side opposite a rectangular dispenser opening having a top edge and a bottom edge with at least one tab on both the top edge and the bottom edge, said tabs being bent outward from the opening and at least one tab having a blade to clip a corner of a packaged bandage upon one by one manual removal of packaged bandage from the dispenser, and;
in the housing a spring and a pusher plate having a spring side and a bandage side where the spring is between the interior side of the housing rear end and the spring side of the pusher plate to apply pressure through the bandage side of the pusher plate to packaged bandages loaded in the dispenser between the bandage side of the pusher plate and the rectangular dispenser opening, and;
where the next available bandage at the dispenser opening is held in a convex shape at the tabs at the top and bottom edge of the dispenser opening.
